(19) United States
(12) Patent Application Publication
(10) Pub. No.: US 2023/0210355 A1
(43) Pub. Date: Jul. 6, 2023

(54) ENDOSCOPE CONDUIT CLEANING TOOL AND ENDOSCOPE SYSTEM

(71) Applicant: OLYMPUS CORPORATION, Tokyo (JP)

(72) Inventor: Koji YAMAYA, Tokyo (JP)

(73) Assignee: OLYMPUS CORPORATION, Tokyo (JP)

(21) Appl. No.: 18/095,045

(22) Filed: Jan. 10, 2023

Related U.S. Application Data
(63) Continuation of application No. PCT/JP2020/027369, filed on Jul. 14, 2020.

(51) Int. Cl. A61B 1/12

(52) U.S. Cl. CPC A61B 1/125; A61B 1/122

(57) ABSTRACT

A conduit cleaning tool includes a long shaft. The shaft includes a linear first part and a second part arranged on a proximal end side with respect to the first part. The second part includes a plurality of bent portions bent to a corrugated shape in a predetermined direction with respect to a longitudinal direction N, and the plurality of bent portions are configured to rub an inner surface of the conduit.

### CROSS-REFERENCE TO RELATED APPLICATION

This application is a continuation application of PCT/JP2020/027369 filed on Jul. 14, 2020, the entire contents of which are incorporated herein by this reference.

### BACKGROUND OF THE INVENTION

### 1. Field of the Invention

The present invention relates to an endoscope conduit cleaning tool and an endoscope system, and the endoscope conduit cleaning tool includes a long shaft having an elasticity, the long shaft being configured to be inserted into a conduit of an endoscope so as to be advanceable/retractable and rotatable in the conduit.

### 2. Description of the Related Art

Endoscopes for use in medical fields are used by being inserted into a body cavity for the purpose of examination and treatment, in particular. Therefore, after use, cleaning processing, disinfecting processing, and sterilization processing are required to be performed on the endoscopes for re-use.

In addition, endoscopes after use are required, such that cleaning and disinfecting processing is surely performed not only on an outer surface of the endoscopes but also on insides of a suction conduit, a gas feeding conduit, and a liquid feeding conduit in the endoscopes. In particular, inner surfaces of the various conduits are required to be scrubbed.

In view of the above, Japanese Patent Application Laid-Open Publication No. 2002-119462 discloses a configuration in which an inside of a conduit whose inner diameter varies can be scrubbed by using one endoscope conduit cleaning tool.

Specifically, the publication discloses a configuration of an endoscope conduit cleaning tool including a brush shaft and a brush member, in which the brush shaft is connected to a distal end of a shaft of the endoscope conduit cleaning tool in a longitudinal direction and is formed in a spiral shape with an elastic material, and the brush member is made of a bristle material or a sponge material, provided on the brush shaft over at least one pitch or more of the spiral shape, and configured to be in contact with an inner surface of the conduit. The publication also discloses a configuration of endoscope conduit cleaning tool including only a brush member.

### SUMMARY OF THE INVENTION

An endoscope conduit cleaning tool according to one aspect of the present invention includes a long shaft having elasticity, the shaft being configured to be inserted into a conduit of an endoscope so as to be advanceable and retractable, and rotatable with respect to the conduit. The shaft includes: a first part arranged on a distal end side of the shaft in a longitudinal direction of the shaft, and formed in a linear shape so as to extend along the longitudinal direction; and a second part arranged on a proximal end side of the shaft in the longitudinal direction with respect to the first part, and including a plurality of bent portions bent to a corrugated shape in a predetermined direction with respect to the longitudinal direction, the plurality of bent portions being configured to rub an inner surface of the conduit.

Furthermore, an endoscope system according to one aspect of the present invention includes the endoscope conduit cleaning tool, and the endoscope including the conduit into which the endoscope conduit cleaning tool is configured to be insertable.

### BRIEF DESCRIPTION OF THE DRAWINGS

FIG. 1 is a view schematically showing a configuration of an endoscope system according to a first embodiment.

FIG. 2 is a partial perspective view showing, in an enlarged manner, the part enclosed by the line II in an endoscope in the endoscope system in FIG. 1.

FIG. 3 is a partial perspective view showing a state where a distal end cover is detached from a distal end portion of an insertion portion of the endoscope in FIG. 2.

FIG. 4 is a cross-sectional view of a distal end side of the endoscope and the distal end cover, taken along the line IV-IV in FIG. 2.

FIG. 5 is a cross-sectional view showing a state where the distal end cover is detached from the distal end portion in FIG. 4.

FIG. 6 is an enlarged perspective view of a conduit cleaning tool in FIG. 1.

FIG. 7 is a side view of the conduit cleaning tool in FIG. 6, which is viewed in the direction of VII in FIG. 6.

FIG. 8 is a partial cross-sectional view showing a state where, instead of the distal end cover, an insertion assisting instrument is mounted to a distal end rigid member in FIG. 3.

FIG. 9 is a partial cross-sectional view of the distal end side of the insertion portion of the endoscope and the insertion assisting instrument, taken along the line IX-IX in FIG. 8.

FIG. 10 is a partial cross-sectional view showing a state where a gas/liquid feeding switching button is removed from a fitting hole of an operation portion of the endoscope in FIG. 1, and the conduit cleaning tool in FIG. 6 is inserted from a side of a fluid discharge groove through a gas feeding conduit or a liquid feeding conduit to the fitting hole of the operation portion.

FIG. 11 is a partial cross-sectional view showing a state where a shaft of the conduit cleaning tool is inserted through the gas feeding conduit in FIG. 10.

FIG. 12 is a partial cross-sectional view showing a modification of a merging configuration of a distal end of the gas feeding conduit and a distal end of the liquid feeding conduit in FIG. 10

FIG. 13 is a partial cross-sectional view of the distal end side of the insertion portion of the endoscope, the insertion assisting instrument, and the shaft, which are enclosed by the line XIII in FIG. 12.

FIG. 14 is a perspective view of a conduit cleaning tool according to a second embodiment.

FIG. 15 is a side view of the conduit cleaning tool in FIG. 14, which is viewed in the direction of XV in FIG. 14.

FIG. 16 is a front view of the conduit cleaning tool in FIG. 14, which is viewed in the direction of XVI in FIG. 14.

FIG. 17 is a front view showing a modification in which a plurality of bent portions, which are formed in a second part of the shaft in FIG. 14, are formed to be corrugated in directions of a plurality of planes.

FIG. 18 is a top view of a conduit cleaning tool according to a third embodiment.

FIG. 19 is a side view of the conduit cleaning tool in FIG. 18, which is viewed in the direction of XIX in FIG. 18.

FIG. 20 is a front view of the conduit cleaning tool in FIG. 19, which is viewed in the direction of IIX in FIG. 19.

FIG. 21 is a partial top view showing a modification of the conduit cleaning tool in FIG. 18.

FIG. 22 is a side view of the conduit cleaning tool in FIG. 21, which is viewed in the direction of IIXII in FIG. 21.

FIG. 23 is a front view of the conduit cleaning tool in FIG. 22, which is viewed in the direction of IIXIII in FIG. 22.

FIG. 24 is a partial cross-sectional view showing a state where the conduit cleaning tool in FIG. 21 is inserted through a gas feeding conduit or a liquid feeding conduit of an endoscope.

FIG. 25 is an enlarged cross-sectional view of the part enclosed by the line IIXV in FIG. 24.

### DETAILED DESCRIPTION OF THE PREFERRED EMBODIMENT(S)

Hereinafter, embodiments of the present invention will be described with reference to drawings.

### First Embodiment

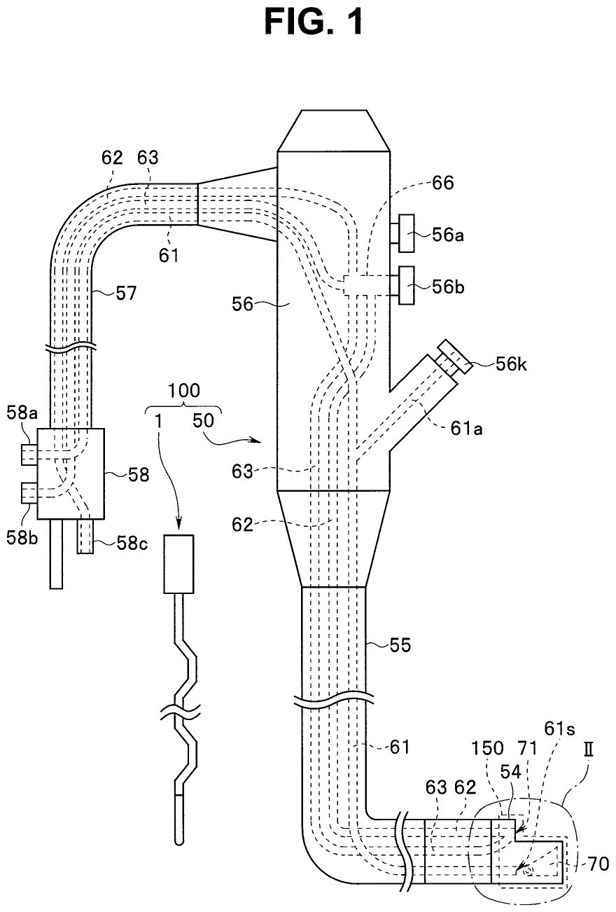

FIG. 1 is a view schematically showing a configuration of an endoscope system according to the present embodiment.

As shown in FIG. 1, an endoscope system 100 has a main part configured by including an endoscope conduit cleaning tool (hereinafter, just referred to as a conduit cleaning tool) 1 and an endoscope 50.

The endoscope 50 has a main part configured by including: an insertion portion 55 formed in an elongated shape along a longitudinal axis direction and configured to be inserted into a body cavity; an operation portion 56 provided continuously with a proximal end of the insertion portion 55; a universal cord 57 extended from the operation portion 56; and a connector 58 provided at an extension end of the universal cord 57.

The insertion portion 55 includes, on a distal end side thereof, a distal end portion 54 in which an image pickup unit, an illumination unit, and the like, not shown, are provided.

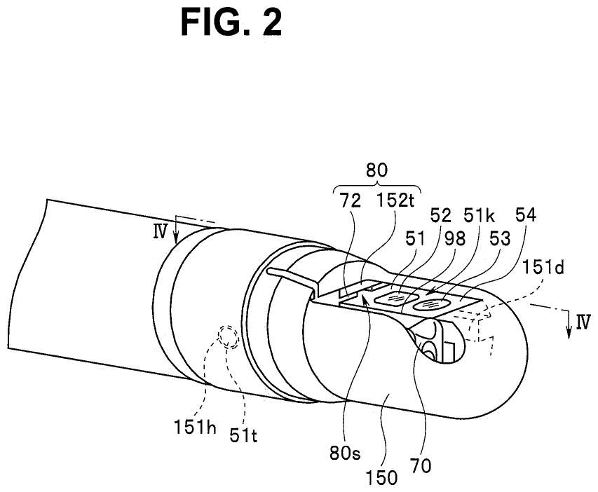

The distal end portion 54 has an outer circumferential surface, a part of which is cut out, and an observation window 52 and an illumination window 53 (both are shown in FIG. 2), which are described later, are exposed on the cutout part. In other words, the endoscope 50 is a side-view endoscope configured to observe lateral sides with respect to the longitudinal axis direction of the insertion portion 55. Note that the distal end portion 54 is configured such that a distal end cover 150 is attachable to and detachable from the distal end portion 54.

In addition, a treatment instrument insertion channel 61 serving also as a suction conduit, a gas feeding conduit 62, and a liquid feeding conduit 63 are provided in insides of the insertion portion 55, the operation portion 56, the universal cord 57, and the connector 58.

A distal end of the channel 61 is open as a distal end opening 61s so as to face a known treatment instrument raising base (forceps elevator) 70 provided in the distal end portion 54, and a proximal end of the channel 61 communicates with a suction pipe sleeve 58a provided to the connector 58. Note that a suction pump, not shown, is configured to be attachable to and detachable from the suction pipe sleeve 58a.

A conduit 61a branched from the channel 61 at a position in the operation portion 56 communicates with a treatment instrument insertion pipe sleeve 56k provided at the operation portion 56.

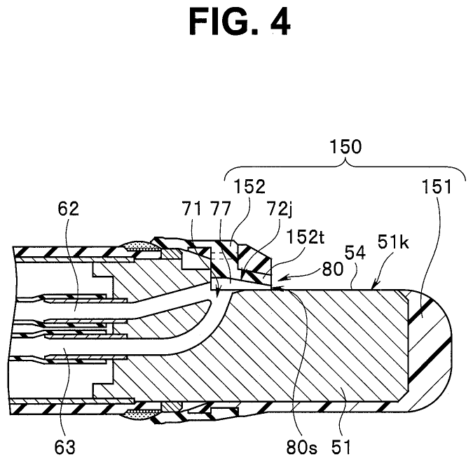

A distal end of the gas feeding conduit 62 and a distal end of the liquid feeding conduit 63 merge with each other. As shown in FIG. 4 to be described later, the merged portion is exposed as a distal end opening 71 in a fluid discharge groove 77 of a fluid discharge member 72 which is provided on a cutout surface 51k of a distal end rigid member 51 constituting the distal end portion 54.

In addition, a proximal end of the gas feeding conduit 62 communicates with a gas supplying pipe sleeve 58b provided to the connector 58. Note that a fluid supplying apparatus, not shown, is configured to be attachable to and detachable from the gas supplying pipe sleeve 58b.

A proximal end of the liquid feeding conduit 63 communicates with a liquid supplying pipe sleeve 58c provided to the connector 58. Note that a liquid supplying apparatus, not shown, is configured to be attachable to and detachable from the liquid supplying pipe sleeve 58c.

Furthermore, the operation portion 56 is provided with a suction switching button 56a and a gas/liquid feeding switching button 56b.

The suction switching button 56a is operated by an operator, when sucking a solid matter, a liquid, and the like in a body cavity through the channel 61.

The gas/liquid feeding switching button 56b is configured to be attachable to and detachable from a fitting hole 66 provided in the gas feeding conduit 62 and the liquid feeding conduit 63 so as to be located at a halfway position in the operation portion.

In addition, the gas/liquid feeding switching button 56b is operated by the operator to switch between gas and liquid when supplying either the gas or the liquid to the observation window 52 and the illumination window 53 (see FIG. 2), which are described later, through the gas feeding conduit 62.

Note that the structures of the respective switching buttons, i.e., the suction switching button 56a and the gas/liquid feeding switching button 56b are the same as those of well-known ones, and therefore detailed descriptions thereof will be omitted.

Next, description will be made on the configurations of the distal end portion 54 and the distal end cover 150, with reference to FIGS. 2 to 5.

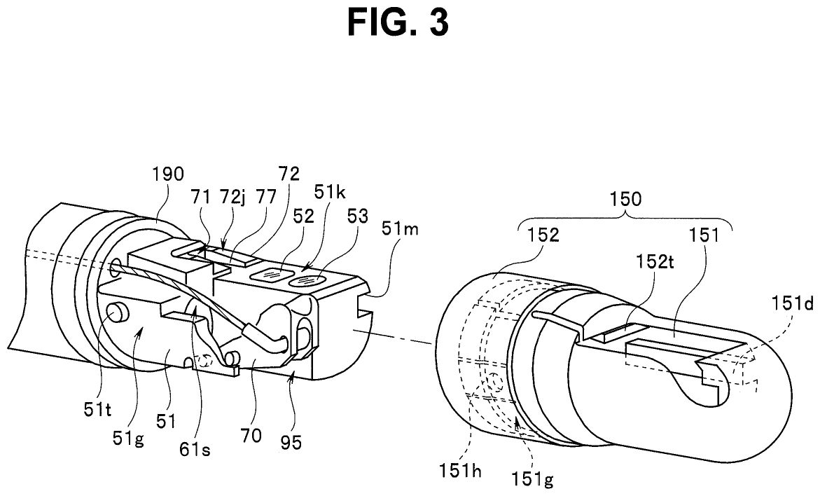

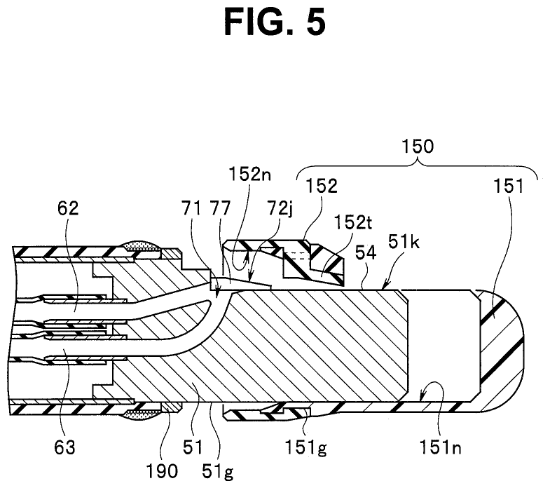

FIG. 2 is a partial perspective view showing, in an enlarged manner, the part enclosed by the line II in the endoscope in the endoscope system in FIG. 1. FIG. 3 is a partial perspective view showing a state where a distal end cover is detached from the distal end portion of the endoscope in FIG. 2. FIG. 4 is a cross-sectional view of the distal end side of the insertion portion of the endoscope and the distal end cover, taken along the line IV-IV in FIG. 2. FIG. 5 is a cross-sectional view showing a state where the distal end cover is detached from the distal end portion in FIG. 4.

As shown in FIGS. 2 to 5, the distal end cover 150 is configured to be attachable to and detachable from the distal end portion 54.

As shown in FIGS. 2 to 5, the distal end portion 54 includes the distal end rigid member 51. On the cutout surface 51k formed on an outer circumferential surface 51g of the distal end rigid member 51, the observation window 52, the illumination window 53, a side surface opening 98 of a treatment instrument raising base disposing space 95, and the fluid discharge member 72 are provided.

As shown in FIG. 2 and FIG. 3, the known treatment instrument raising base 70 is provided in the treatment instrument raising base disposing space 95. The distal end opening 61s of the channel 61 is formed at a position on the proximal end side with respect to the treatment instrument raising base 70 in the distal end rigid member 51, so as to face the treatment instrument raising base 70.

The treatment instrument raising base 70 is configured to change an advancing direction of each of various treatment instruments toward the side surface opening 98. Each of the various treatment instruments is inserted from the treatment instrument insertion pipe sleeve 56k into the conduit 61a and the channel 61, to be protruded from the distal end opening 61s.

As shown in FIG. 3, the fluid discharge member 72 is formed in a tapered shape such that the thickness of a top surface 72j becomes smaller toward the distal end side.

In addition, the fluid discharge member 72 includes the fluid discharge groove 77 which is configured to open on the distal end of the fluid discharge member 72 opposed to the observation window 52 and on the top surface 72j. The fluid discharge groove 77 is a fluid discharge port communicating with the above-described distal end opening 71. Note that the fluid discharge member 72 may be formed integrally with the distal end rigid member 51.

Various kinds of fluids discharged from the distal end opening 71 are discharged through the fluid discharge groove 77.

Furthermore, as shown in FIG. 2 and FIG. 3, a locking pin 51t is formed on the outer circumferential surface 51g of the distal end rigid member 51.

The locking pin 51t is configured to be locked in a locking hole 151h, to be described later, of a first cover 151, as shown in FIG. 2, when the distal end cover 150 is mounted on the outer circumferential surface 51g of the distal end rigid member 51.

In addition, as shown in FIG. 3, a locking groove 51m extending along the longitudinal axis direction is formed on the outer circumferential surface 51g of the distal end rigid member 51.

The locking groove 51m is configured such that a locking protrusion 151d, to be described later, of the first cover 151 is engaged with (fitted in) the locking groove, as shown in FIG. 2, when the distal end cover 150 is mounted on the outer circumferential surface 51g of the distal end rigid member 51.

As shown in FIGS. 3 to 5, the distal end cover 150 is configured of the first cover 151 and a second cover 152.

The first cover 151 is made of a rigid resin such as plastic, and formed in a short cylindrical shape along the longitudinal axis direction so as to have a cap shape whose distal end is closed. The first cover 151 is configured such that an inner circumferential surface 151n is engageable with the outer circumferential surface 51g of the distal end rigid member 51.

Furthermore, as shown in FIG. 3, the locking protrusion 151d provided on the inner circumferential surface 151n of the first cover 151 so as to be along the longitudinal axis direction.

The locking protrusion 151d is configured to engage with the locking groove 51m, when the first cover 151 is engaged with the outer circumferential surface 51g of the distal end rigid member 51. With this, the first cover 151 is positioned in the rotational direction with respect to the distal end rigid member 51.

In addition, as shown in FIG. 3, a circumferential groove is formed on the outer circumferential surface 151g of the first cover 151, and in the circumferential groove, the locking hole 151h penetrating the outer circumferential surface 151g and the inner circumferential surface 151n is formed.

The locking pin 51t is locked in the locking hole 151h, when the first cover 151 is engaged with the outer circumferential surface 51g of the distal end rigid member 51. With this, the first cover 151 is positioned in the rotational direction and in the longitudinal axis direction with respect to the distal end rigid member 51.

The second cover 152 is configured by including a member, such as rubber, for example, which is more elastic than the first cover 151, and formed in a short cylindrical shape along the longitudinal axis direction.

Furthermore, the second cover 152 is configured to be engageable with the first cover 151 such that a part of an inner circumferential surface 152n closely contacts the outer circumferential surface 151g of the first cover 151 and to be engageable with the distal end rigid member 51 such that a proximal end side portion of the inner circumferential surface 152n in the longitudinal axis direction closely contacts an insulation ring 190 disposed on a proximal end side of the outer circumferential surface 51g of the distal end rigid member 51.

Note that the second cover 152 is made of rubber in order to ensure the water-tightness and the electric insulation property of the distal end rigid member 51 when the distal end cover 150 is mounted on the outer circumferential surface 51g.

In addition, the second cover 152 includes, at the distal end thereof, a protruding piece 152t having a bottom surface formed in a shape tapered in a reduced-diameter direction toward the distal end side. Note that the bottom surface of the protruding piece 152t is formed so as to have the same length as that of the top surface 72j of the fluid discharge member 72.

The bottom surface of the protruding piece 152t closely contacts the top surface 72j of the fluid discharge member 72 when the distal end cover 150 is mounted on the distal end rigid member 51. In other words, the protruding piece 152t covers the top surface 72j, to thereby cover the top surface of the fluid discharge groove 77.

As a result, as shown in FIG. 2 and FIG. 4, a nozzle 80 is formed by the protruding piece 152t and the fluid discharge member 72. The nozzle 80 has a distal end 80s which opens as a discharge port, and is configured to supply fluid from the distal end opening 71 and the fluid discharge groove 77 to the observation window 52 and the illumination window 53.

Note that, in the above-described description, the second cover 152 is configured to be engageable with the outer circumferential surface 151g of the first cover 151. However, actually, after the second cover 152 is engaged with the outer circumferential surface 151g of the first cover 151, the second cover 152 may be held in the engaged state, or the first cover 151 and the second cover 152 may be brought into an inseparable state by adhesion.

The first cover 151 and the second cover 152 may be formed integrally as the distal end cover 150.

As described above, the insertion portion 55 is inserted into a body cavity, with the distal end cover 150 being mounted on the distal end portion 54.

After that, when it is hard to continue observation due to the dirt on the observation window 52, the operator operates the gas/liquid feeding switching button 56b, to thereby cause the fluid fed through the gas feeding conduit 62 or the liquid feeding conduit 63 to be sprayed from the nozzle 80 to the observation window 52 to remove the dirt. As a result, a field of view from the observation window 52 is ensured.

Note that it has been described that the distal end cover 150 is also attachable to and detachable from the outer circumferential surface 51g of the distal end rigid member 51. However, actually, before the use of the endoscope, the distal end cover 150 is fixed to the distal end rigid member 51 by the engagement between the locking hole 151h and the locking pin 51t. Therefore, after the use of the endoscope, when the distal end cover 150 is detached from the distal end rigid member 51, the distal end cover 150 is destroyed to be detached from the distal end rigid member 51. After that, the distal end cover 150 is discarded, and then cleaning, disinfecting, and sterilizing processing is performed on the endoscope 50.

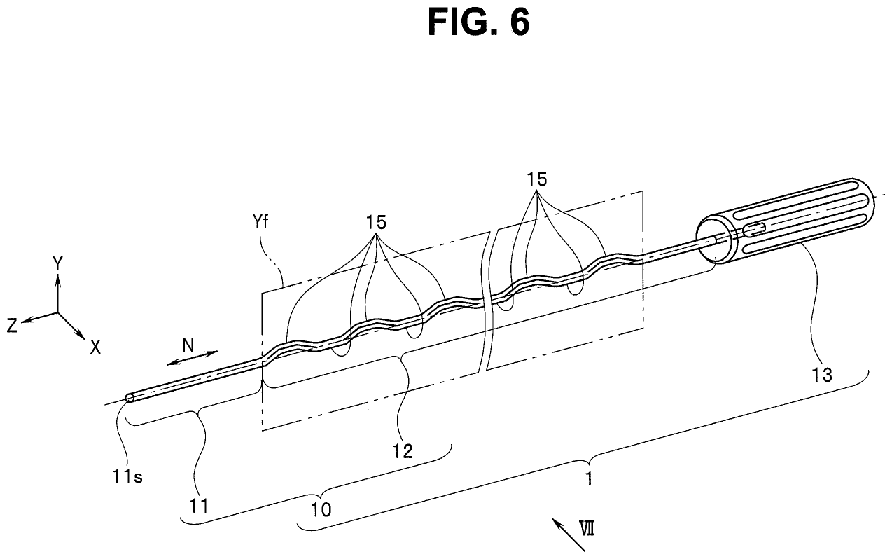

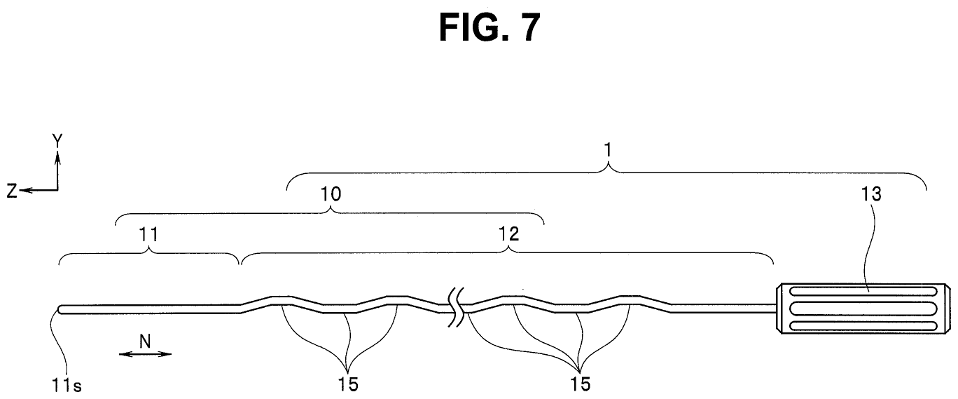

Next, description will be made on the configuration of the conduit cleaning tool 1 with reference to FIG. 6 and FIG. 7. FIG. 6 is an enlarged perspective view of the conduit cleaning tool in FIG. 1. FIG. 7 is a side view of the conduit cleaning tool in FIG. 6, which is viewed in the direction of VII in FIG. 6.

As shown in FIG. 6 and FIG. 7, the conduit cleaning tool 1 is configured of a shaft 10 and a grasping portion 13.

The shaft 10 is inserted through the conduit of the endoscope 50, for example, at least one of the gas feeding conduit 62 and the liquid feeding conduit 63, so as to be advanceable/retractable, and rotatable. The shaft 10 is made of a material having elasticity and formed in a long shape.

Specifically, the shaft 10 is made of an elastic material such as polypropylene, polyamide, nylon, fluorocarbon, polyethylene, polyester, or the like, and formed to be solid and to have an outer diameter of approximately 1 mm. The shaft 10 is formed to have a length substantially equal to a length obtained by adding the length of the operation portion 56 to the length of the insertion portion 55 of the endoscope 50.

The grasping portion 13 is provided so as to be continuous with the proximal end of the shaft 10 in the longitudinal direction N. The grasping portion 13 is a part to be grasped by the operator when the operator inserts the shaft 10 selectively into the gas feeding conduit 62 or the liquid feeding conduit 63. Note that it is preferable that the grasping portion 13 is made of the same material as that of the shaft 10, but may be made of a material different from that of the shaft 10.

The shaft 10 is configured of a first part 11 and a second part 12.

The first part 11 is arranged on the distal end side of the shaft 10 in the longitudinal direction N, and formed in a linear shape so as to extend along the longitudinal direction N.

Specifically, the first part 11 is formed in the length of about 10 mm to 50 mm in the longitudinal direction N.

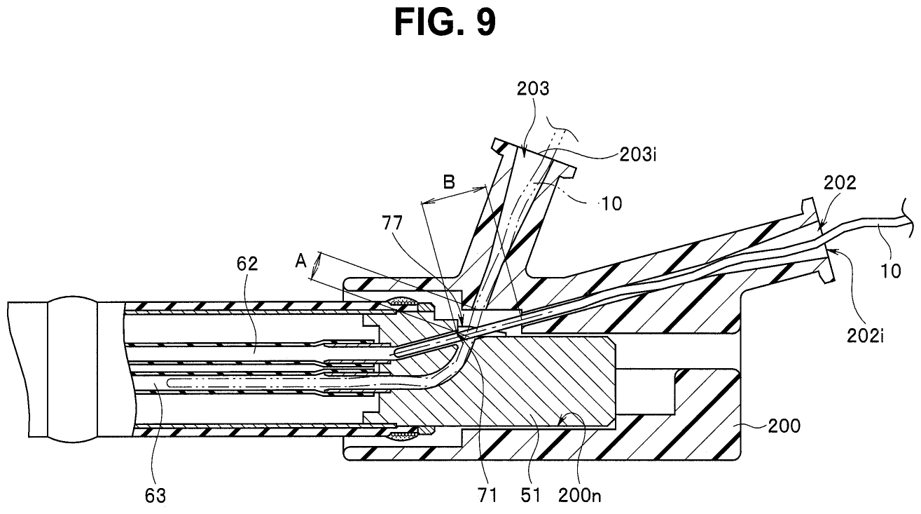

If the first part 11 is formed in a length shorter than the above-described length, it would be difficult to guide a distal end 11s of the first part 11 smoothly into the gas feeding conduit 62 or the liquid feeding conduit 63, when the shaft 10 is inserted selectively into the gas feeding conduit 62 or the liquid feeding conduit 63 by means of an insertion assisting instrument 200, as shown in FIG. 9 to be described later.

In addition, as shown in FIG. 7, the distal end 11s of the first part 11 is formed in a rounded shape so as not to damage the inside of the conduit and not to be caught by a step in the conduit, when the shaft 10 is inserted through the gas feeding conduit 62 or the liquid feeding conduit 63. Note that, for the same purposes as described above, a structure having a rounded shape such as a spherical shape or a hemispherical shape may be additionally fixed to the distal end 11s.

The second part 12 is arranged on the proximal end side in the longitudinal direction N with respect to the first part 11, and includes a plurality of bent portions 15 bent to a corrugated shape in a predetermined direction with respect to the longitudinal direction N.

Specifically, the plurality of bent portions 15 are shaped to displace the shaft 10 in the predetermined direction so as to be corrugated with a predetermine depth (corrugation depth) in at least one plane. Specifically, in the present embodiment, as shown in FIG. 6, when the longitudinal direction N is assumed to be a Z direction, the plurality of bent portions 15 are shaped to displace the shaft 10 so as to be corrugated with the predetermined depth, in a plane Yf in a Y direction orthogonal to the Z direction.

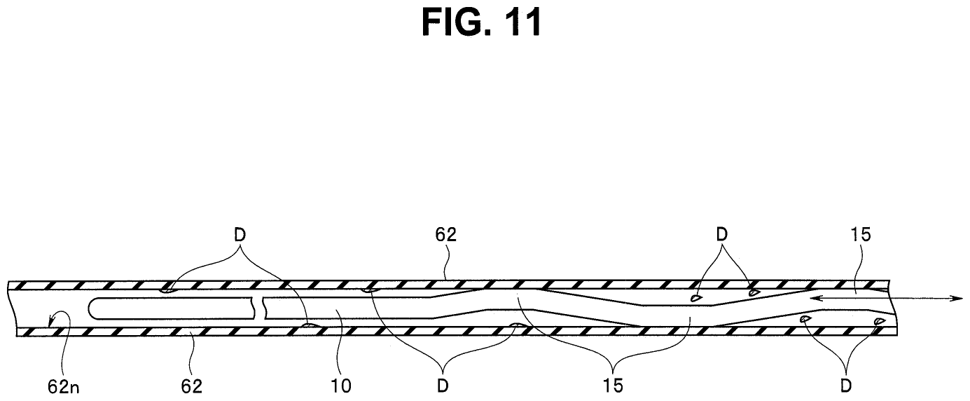

When the shaft 10 is inserted through the gas feeding conduit 62 or the liquid feeding conduit 63, the plurality of bent portions 15 contact the inner surface of the conduit, to rub the inner surface by the shaft 10 being advanced/retracted, or rotated, to thereby remove the dirt D (see FIG. 11) adhered on the inner surface.

Therefore, the corrugation depth of the plurality of bent portions 15 is substantially equal to the inner diameter of the gas feeding conduit 62 or the liquid feeding conduit 63 such that the plurality of bent portions 15 surely contact the inner surface of the conduit.

Note that if the maximum value of the corrugation depth of the plurality of bent portions 15 is set to be larger than the maximum inner diameter over the entire length of the gas feeding conduit 62 or the liquid feeding conduit 63, the plurality of bent portions 15 can be surely brought into contact with the inner surface of the conduit even if the inner diameter of the conduit partially varies in the conduit.

In addition, FIG. 6 and FIG. 7 show the case where the corrugated shape that constitutes the plurality of bent portions 15 is formed of straight lines. However, the corrugated shape may be formed of curved lines.

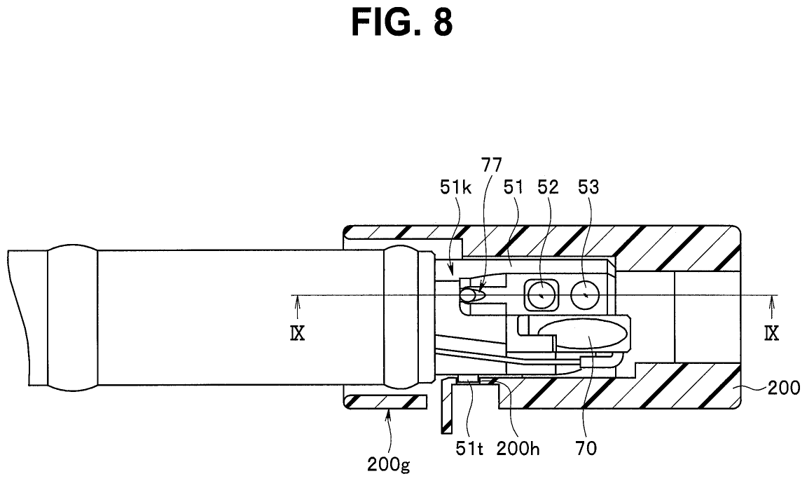

Next, description will be made on the method of inserting the conduit cleaning tool 1 thus configured through the gas feeding conduit 62 or the liquid feeding conduit 63, with reference to FIGS. 8 to 11.

FIG. 8 is a partial cross-sectional view showing a state where, instead of the distal end cover, the insertion assisting instrument is mounted to a distal end rigid member in FIG. 3. FIG. 9 is a partial cross-sectional view of the distal end side of the insertion portion of the endoscope and the insertion assisting instrument, taken along the line IX-IX in FIG. 8.

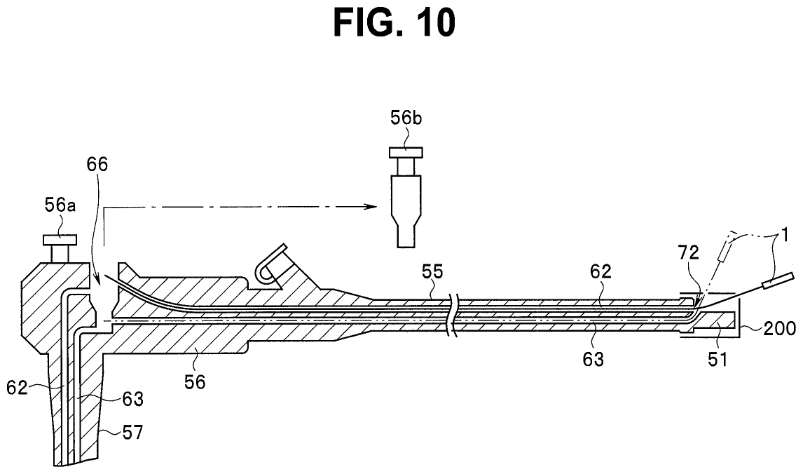

In addition, FIG. 10 is a partial cross-sectional view showing a state where the gas/liquid feeding switching button is removed from the fitting hole of the operation portion of the endoscope in FIG. 1, and the conduit cleaning tool in FIG. 6 is inserted from the side of the fluid discharge groove through the gas feeding conduit or the liquid feeding conduit to the fitting hole of the operation portion. FIG. 11 is a partial cross-sectional view showing a state where the shaft of the conduit cleaning tool is inserted through the gas feeding conduit in FIG. 10.

When cleaning the endoscope 50 with the distal end cover 150 being mounted on the distal end portion 54 after use, first the operator removes the distal end cover 150 from the distal end portion 54, as described above. As a result, the fluid discharge groove 77 is exposed, as shown in FIG. 3 and FIG. 5.

Next, as shown in FIG. 8, the operator mounts the insertion assisting instrument 200 to the distal end rigid portion 51 at the distal end portion 54.

Specifically, as shown in FIG. 8 and FIG. 9, the insertion assisting instrument 200 is formed in a shape of a short cylinder along the longitudinal axis direction so as to have a cap shape whose distal end is closed, and configured such that an inner circumferential surface 200n is engageable with the outer circumferential surface 51g of the distal end rigid member 51.

In addition, as shown in FIG. 8, on an outer circumferential surface 200g of the insertion assisting instrument 200, an locking hole 200h penetrating through the outer circumferential surface 200g and the inner circumferential surface 200n is formed.

The locking pin 51t is locked in the locking hole 200h, when the insertion assisting instrument 200 is engaged with the outer circumferential surface 51g of the distal end rigid member 51. With this, the insertion assisting instrument 200 is positioned in the rotational direction and the longitudinal axis direction with respect to the distal end rigid member 51.

In addition, as shown in FIG. 9, the insertion assisting instrument 200 includes an insertion path for gas feeding conduit 202 and an insertion path for liquid feeding conduit 203, each of which communicates with the fluid discharge groove 77 when the insertion assisting instrument 200 is mounted to the distal end rigid member 51. The insertion path for gas feeding conduit 202 and the insertion path for liquid feeding conduit 203 are formed at angles different from each other.

Specifically, the angle of the insertion path for gas feeding conduit 202 with respect to the longitudinal axis of the distal end rigid member 51 coincides with the angle at which the distal end side of the gas feeding conduit 62 faces the distal end opening 71. The angle of the insertion path for liquid feeding conduit 203 with respect to the longitudinal axis of the distal end rigid member 51 coincides with the angle at which the distal end side of the liquid feeding conduit 63 faces the distal end opening 71.

The insertion path for gas feeding conduit 202 is used when the shaft 10 of the conduit cleaning tool 1 is inserted from an insertion port 202i into the gas feeding conduit 62 through the fluid discharge groove 77 and the distal end opening 71.

In addition, as shown in FIG. 9, when the insertion assisting instrument 200 is mounted to the distal end rigid member 51, a straight-line distance B between the distal end opening 71 and an end portion of the insertion path for gas feeding conduit 202 is set to be shorter than the first part 11 of the shaft 10.

With such a configuration and the coincidence of the angle of the distal end side of the gas feeding conduit 62 and the angle of the insertion path for gas feeding conduit 202, the first part 11 can be smoothly and easily guided from the distal end opening 71 into the gas feeding conduit 62 without being caught and can be inserted through the gas feeding conduit 62.

The insertion path for liquid feeding conduit 203 is used when the shaft 10 of the conduit cleaning tool 1 is inserted from an insertion port 203i into the liquid feeding conduit 63 through the fluid discharge groove 77 and the distal end opening 71.

In addition, as shown in FIG. 9, when the insertion assisting instrument 200 is mounted to the distal end rigid member 51, a straight-line distance A between the distal end opening 71 and an end portion of the insertion path for liquid feeding conduit 203 is set to be shorter than the first part 11 of the shaft 10.

With such a configuration and the coincidence of the angle of the distal end side of the liquid feeding conduit 63 and the angle of the insertion path for liquid feeding conduit 203, the first part 11 can be smoothly and easily guided from the distal end opening 71 into the liquid feeding conduit 63 without being caught and can be inserted through the liquid feeding conduit 63.

Note that, as described above, the insertion assisting instrument 200 is only intended to enable the shaft 10 to be inserted easily and selectively into the gas feeding conduit 62 or the liquid feeding conduit 63. Therefore, the operator may insert the shaft 10 selectively into the gas feeding conduit 62 or the liquid feeding conduit 63 directly through the fluid discharge groove 77 and the distal end opening 71, without using the insertion assisting instrument 200.

The shaft 10 inserted from the distal end opening 71 into the gas feeding conduit 62 or the liquid feeding conduit 63 is inserted through to reach the fitting hole 66 from which the gas/liquid feeding switching button 56b of the operation portion 56 is removed as shown in FIG. 10.

In the insertion process to reach the fitting hole 66, as shown in FIG. 11, the plurality of bent portions 15 of the shaft 10 contact the inner surface 62n of the gas feeding conduit 62, for example. At this time, the shaft 10 is finely advanced forward and retracted backward, or rotated, and thereby the plurality of bent portions 15, as shown in FIG. 11, remove the dirt D adhered on the inner surface 62n of the gas feeding conduit 62 in the case where the shaft 10 is inserted through the gas feeding conduit 62, for example.

Note that the same is true on the case where the shaft 10 is inserted through the liquid feeding conduit 63.

In addition, in the present embodiment, as shown in FIG. 4, FIG. 5, FIG. 9, and FIG. 10, description is made by taking the case where the distal end of the gas feeding conduit 62 and the distal end of the liquid feeding conduit 63 merge with each other at the distal end opening 71 as an example. However, another merging configuration is also known.

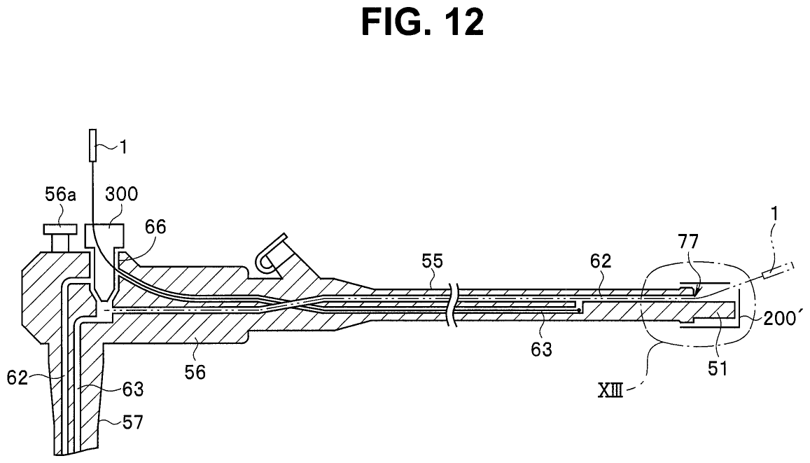

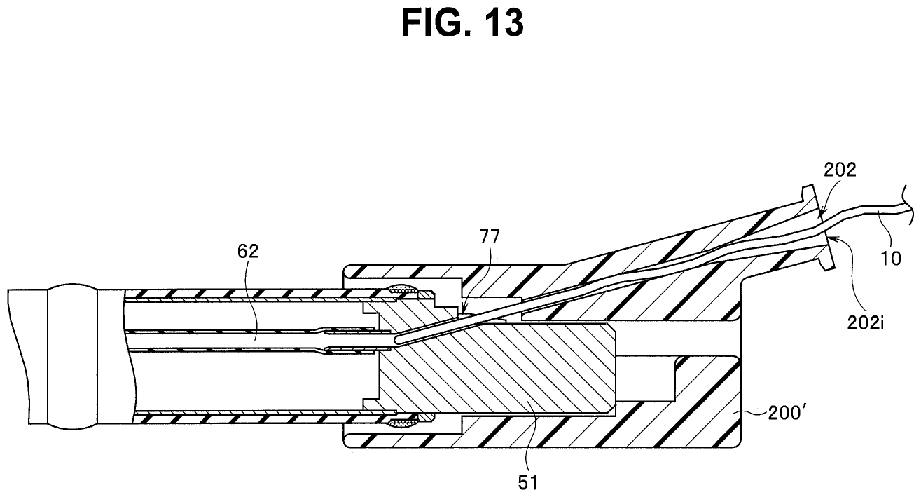

FIG. 12 is a partial cross-sectional view showing a modification of the merging configuration of the distal end of the gas feeding conduit and the distal end of the liquid feeding conduit in FIG. 10. FIG. 13 is a partial cross-sectional view of the distal end side of the insertion portion of the endoscope, the insertion assisting instrument, and the shaft, which are enclosed by the line XIII in FIG. 12.

As shown in FIG. 12, a configuration in which the distal end of the liquid feeding conduit 63 merges with the gas feeding conduit 62 at the halfway position of the insertion portion 55 is known.

Such a configuration has an advantage of reducing the diameter of the distal end portion 54, since the merged portion is not located at the distal end portion 54.

However, in the configuration shown in FIG. 12, the shaft 10 can be inserted into the gas feeding conduit 62 through the fluid discharge groove 77, as described above, but cannot be inserted into the liquid feeding conduit 63.

Therefore, as shown in FIG. 12 and FIG. 13, in the present configuration, the shaft 10 may be inserted into the gas feeding conduit 62 from the fluid discharge groove 77 in the similar manner as in the above-described present embodiment, and the shaft 10 may be inserted into the liquid feeding conduit 63 from the fitting hole 66.

Note that if the insertion assisting instrument is used, as shown in FIG. 12 and FIG. 13, an insertion assisting instrument 200′, which is to be mounted to the distal end rigid member 51, includes only the insertion path for gas feeding conduit 202, and the shaft 10 is inserted from the insertion port 202i into the gas feeding conduit 62 through the insertion path for gas feeding conduit 202 and the fluid discharge groove 77.

In addition, an insertion assisting instrument 300 different from the insertion assisting instrument 200′ is mounted into the fitting hole 66, and the shaft 10 is inserted from an insertion path for liquid feeding conduit formed in the insertion assisting instrument 300 into the liquid feeding conduit 63 through the fitting hole 66.

Thus, in the present embodiment, the conduit cleaning tool 1 configured to clean the inside of the gas feeding conduit 62 or the inside of the liquid feeding conduit 63 includes the flexible shaft 10. The shaft 10 has the plurality of bent portions 15 formed in a corrugated shape to be in contact with the inner surface of the conduit to rub the inner surface.

In such a configuration, since the plurality of bent portions 15 are not formed in a spiral shape as a conventional brush but formed in the corrugated shape, the plurality of bent portions 15 are not likely to be compressed in the longitudinal direction N when the shaft 10 is advanced and retracted in the conduit. The pushing force from the grasping portion 13 is easily transmitted to the distal end 11s of the shaft 10, to thereby provide an excellent insertability.

In addition, the shaft 10, which is to be inserted into the conduit, does not include a rigid portion. Such a configuration not only provides an excellent insertability in the conduit, but also prevents a damage on the conduit even if the plurality of bent portions 15 contact the inner surface of the conduit.

In addition, if the shaft 10 is advanced and retracted in order to remove the dirt D on the inner surface in the conduit, the plurality of bent portions 15 weakly contact the inner surface of the conduit not locally but over the entire length of the conduit. Therefore, the present embodiment enables the dirt D to be removed, without causing the buckling of the conduit by pulling-in operation of the shaft 10 in the above-described conventional example.

In addition, the conduit cleaning tool 1 according to the present embodiment is not provided with a brush member provided in the conventional example, the conduit cleaning tool 1 can be manufactured by integrally forming the shaft 10 in the above-described predetermined shape. Therefore, the conduit cleaning tool 1 can be manufactured as a disposable tool at a low cost.

Thus, it is possible to provide the conduit cleaning tool 1 and the endoscope system 100 that are capable of easily and surely cleaning the entirety of the inside of the conduit without damaging and buckling of the conduit and that can be manufactured at a low cost.

### Second Embodiment

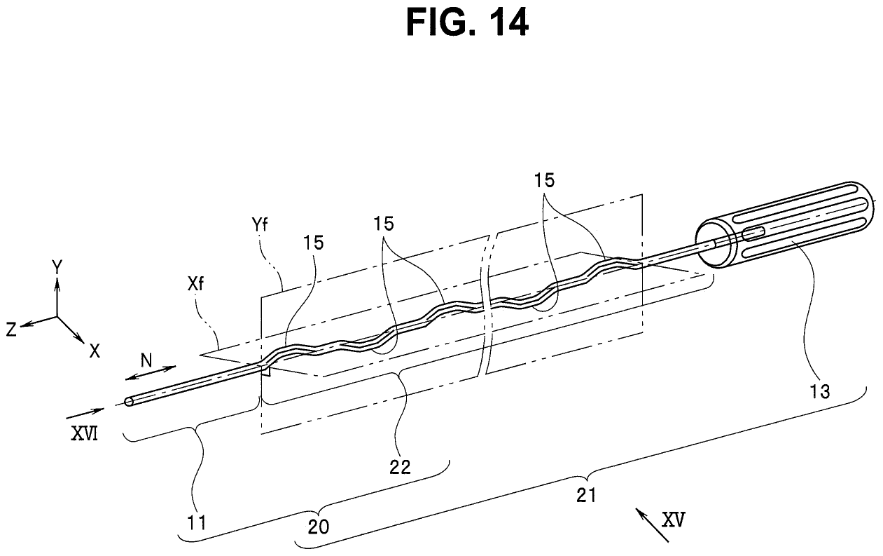

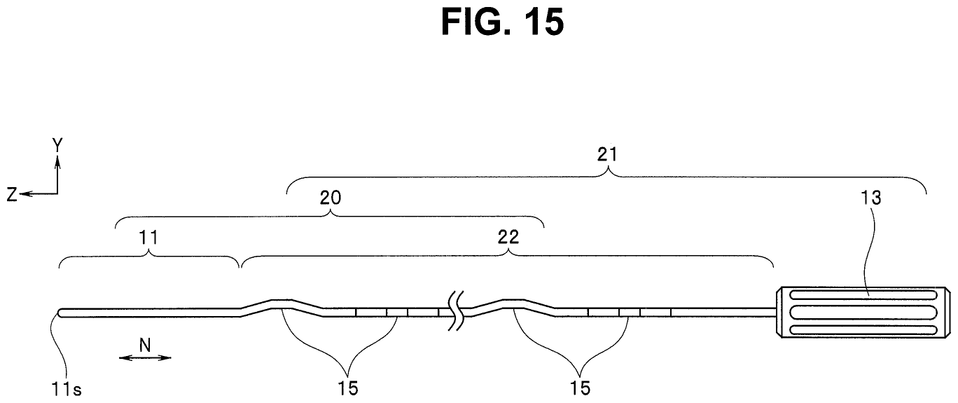

FIG. 14 is a perspective view of a conduit cleaning tool according to the present embodiment. FIG. 15 is a side view of the conduit cleaning tool in FIG. 14, which is viewed in the direction of XV in FIG. 14. FIG. 16 is a front view of the conduit cleaning tool in FIG. 14, which is viewed in the direction of XVI in FIG. 14.

Configurations of the conduit cleaning tool and an endoscope system according to the second embodiment are different in a shape of a plurality of bent portions from those of the conduit cleaning tool and the endoscope system according to the above-described first embodiment shown in FIGS. 1 to 13.

Only such a different point will be described, and the same constituent elements as those in the first embodiment are attached with the same reference signs and descriptions thereof will be omitted.

As shown in FIG. 14 and FIG. 15, a conduit cleaning tool 21 is configured of a shaft 20 and a grasping portion 13.

The shaft 20 is inserted through the conduit of the endoscope 50, for example, at least one of the gas feeding conduit 62 and the liquid feeding conduit 63, so as to be advanceable/retractable and rotatable. The shaft 20 is made of a material having elasticity and formed in a long shape.

Specifically, the shaft 20 is made of an elastic material such as polypropylene, polyamide, nylon, fluorocarbon, polyethylene, polyester, or the like, and formed to be solid and to have an outer diameter of approximately 1 mm. The shaft 20 is formed to have a length substantially equal to a length obtained by adding the length of the operation portion 56 to the length of the insertion portion 55 of the endoscope 50.

In addition, the shaft 20 is configured of a first part 11 and a second part 22.

The second part 22 is arranged on the proximal end side in the longitudinal direction N with respect to the first part 11, and includes a plurality of bent portions 15 bent to a corrugated shape in a predetermined direction with respect to the longitudinal direction N.

Specifically, the plurality of bent portions 15 are shaped to displace the shaft 10 in predetermined directions so as to be corrugated with a predetermined depth in two planes. Specifically, in the present embodiment, as shown in FIGS. 14 to 16, when the longitudinal direction N is assumed to be the Z direction, the plurality of bent portions 15 are shaped to displace the shaft 10 so as to be corrugated with the predetermined depth, in a plane Yf in the Y direction and a plane Xf in the X direction that are orthogonal to the Z direction.

Note that other configurations and working are the same as those in the above-described first embodiment, and such a configuration is also capable of providing the same effects as those in the first embodiment.

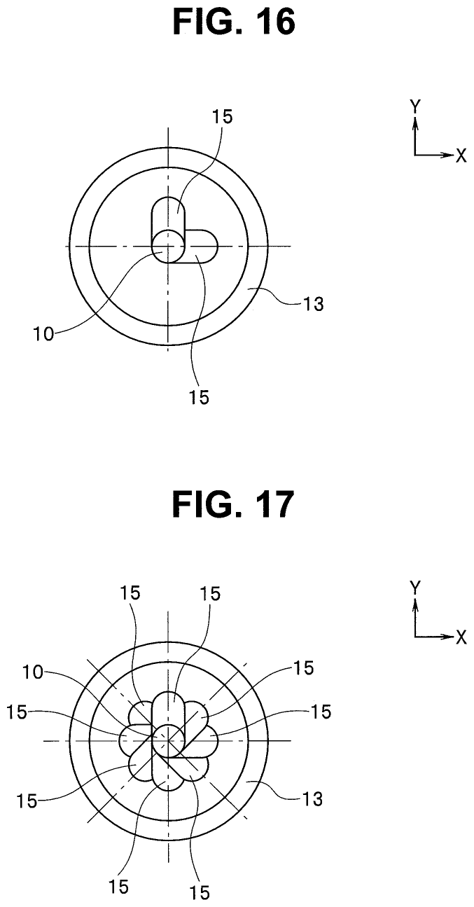

Hereinafter, description will be made on a modification, with reference to FIG. 17. FIG. 17 is a front view of the modification in which the plurality of bent portions, which are formed in the second part of the shaft in FIG. 14, are formed to be corrugated in directions of a plurality of planes.

As shown in FIG. 17, the plurality of bent portions 15 may be formed to be corrugated not only in the direction of one plane as in the first embodiment and in the directions of the two planes in the present embodiment, but also in directions of plurality of planes, i.e., three or more planes.

Note that, in this case, in a circumferential direction of the shaft 10, the plurality of bent portions 15 need not be formed sequentially but may be formed in a random manner.

According to such a configuration, when the shaft 10 is inserted into the above-described conduit, the plurality of bent portions 15 contact the inner surface of the conduit uniformly in the circumferential direction of the shaft 10. Therefore, even if the shaft 10 is not rotated in the conduit, the dirt D on the inner surface of the conduit can be surely removed only by advancing and retracting the shaft 10.

Note that other configurations and effects are the same as those in the above-described present embodiment.

### Third Embodiment

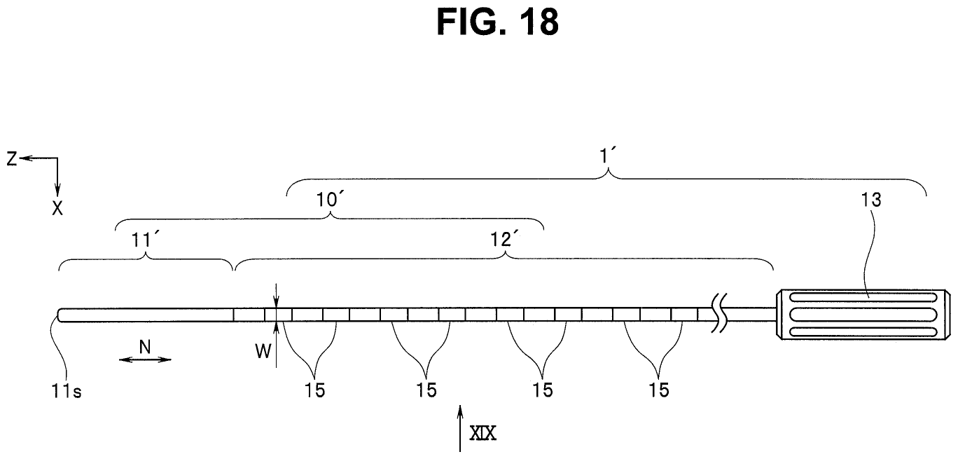

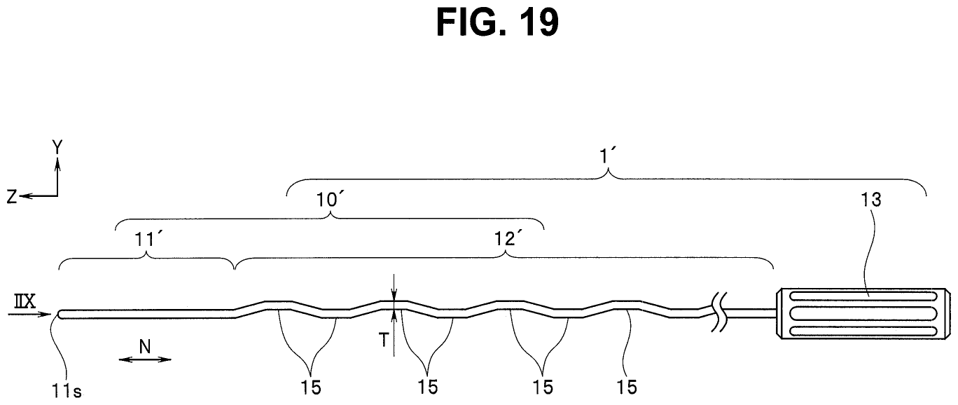

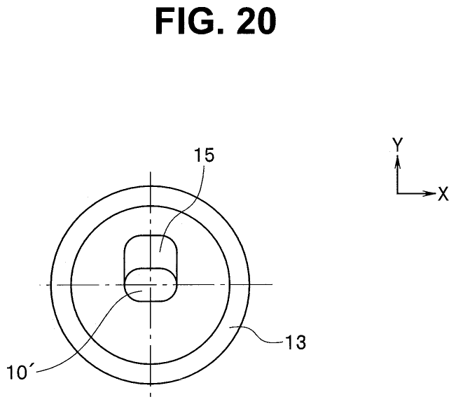

FIG. 18 is a top view of a conduit cleaning tool according to the present embodiment. FIG. 19 is a side view of the conduit cleaning tool in FIG. 18, which is viewed in the direction of XIX in FIG. 18. FIG. 20 is a front view of the conduit cleaning tool in FIG. 19, which is viewed in the direction of IIX in FIG. 19.

Configurations of a conduit cleaning tool and an endoscope system according to the third embodiment differ in a shape of a shaft from that of the above-described conduit cleaning tool and the endoscope system according to the first embodiment shown in FIGS. 1 to 13, and from that of the conduit cleaning tool and the endoscope system according to the second embodiment shown in FIGS. 14 to 17.

Only such a different point will be described, and the same constituent elements as those in the first and second embodiments are attached with the same reference signs and descriptions thereof will be omitted.

As shown in FIG. 18 and FIG. 19, a conduit cleaning tool 1′ is configured of a shaft 10′ and a grasping portion 13.

The shaft 10′ is inserted through the conduit of the endoscope 50, for example, at least one of the gas feeding conduit 62 and the liquid feeding conduit 63, so as to be advanceable/retractable, and rotatable. The shaft 10′ is made of a material having elasticity and formed in a long shape.

Specifically, the shaft 10′ is made of an elastic material such as polypropylene, polyamide, nylon, fluorocarbon, polyethylene, polyester, or the like, and formed to be solid. The shaft 10′ is formed to have a length substantially equal to a length obtained by adding the length of the operation portion 56 to the length of the insertion portion 55 of the endoscope 50.

In addition, the shaft 10′ is configured of a first part 11′ and a second part 12′.

Furthermore, in the present embodiment, as shown in FIGS. 18 to 20, when the longitudinal direction N is assumed to be the Z direction, the shaft 10′ is formed such that a thickness W of the plurality of bent portions 15 in another direction X orthogonal to the Z direction and a bent direction Y is larger than a thickness T of the plurality of bent portions 15 in the bent direction Y orthogonal to the Z direction (W T).

Note that other configurations of the first part 11′ and the second part 12′ are the same as those of the first part 11 and the second part 12 in the first embodiment.

In such a configuration, if both the thickness W and the thickness T are set to be too small, the rigidity of the shaft 10 will be lost, by the plurality of bent portions 15 contacting the inner surface of the conduit a plurality of times as described above.

However, if the sufficient thickness W is ensured in the other direction X as described above, the rigidity of the shaft 10 is improved, and if the thickness T in the bent direction Y is made to be smaller, the plurality of bent portions 15 can be bent more easily, to thereby be capable of improving a property of removing the dirt D on the inner surface of the conduit.

Therefore, the relationship of the thicknesses W>T is not required to be set to the entirety of the shaft 10, but may be set only to the second part 12.

Note that other configurations and working are the same as those in the above-described first and second embodiments, and such a configuration is also capable of providing the same effects as those in the first and second embodiments.

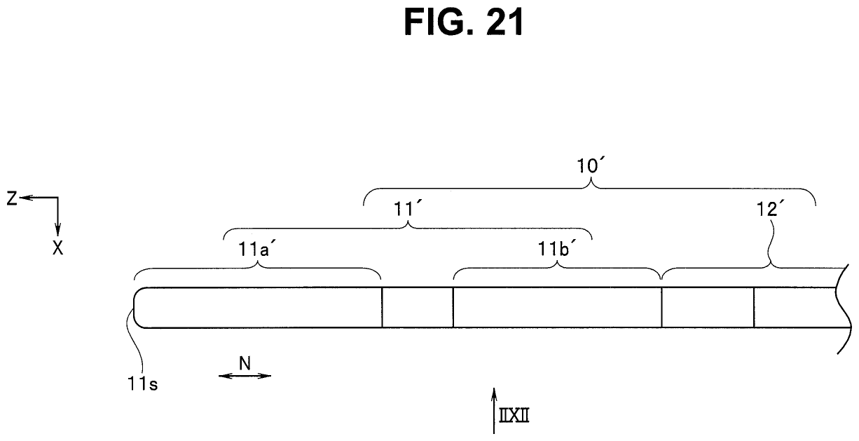

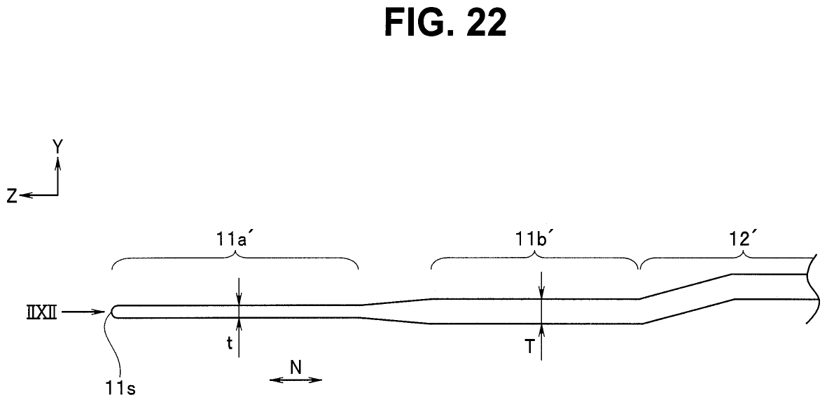

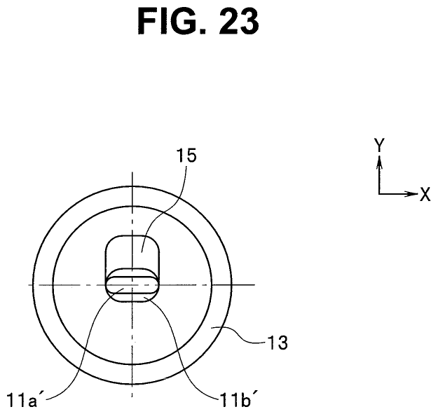

Hereinafter, description will be made on a modification, with reference to FIGS. 21 to 25. FIG. 21 is a partial top view showing the modification of the conduit cleaning tool in FIG. 18. FIG. 22 is a side view of the conduit cleaning tool in FIG. 21, which is viewed in the direction of IIXII in FIG. 21. FIG. 23 is a front view of the conduit cleaning tool in FIG. 22, which is viewed in the direction of IIXIII in FIG. 22.

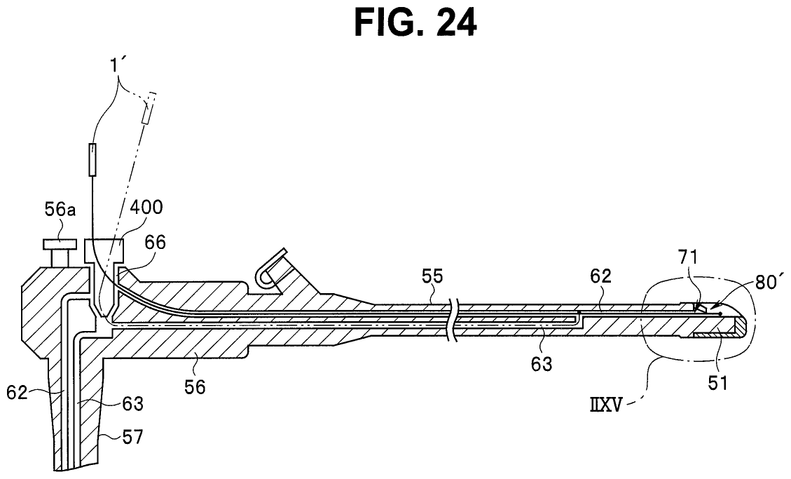

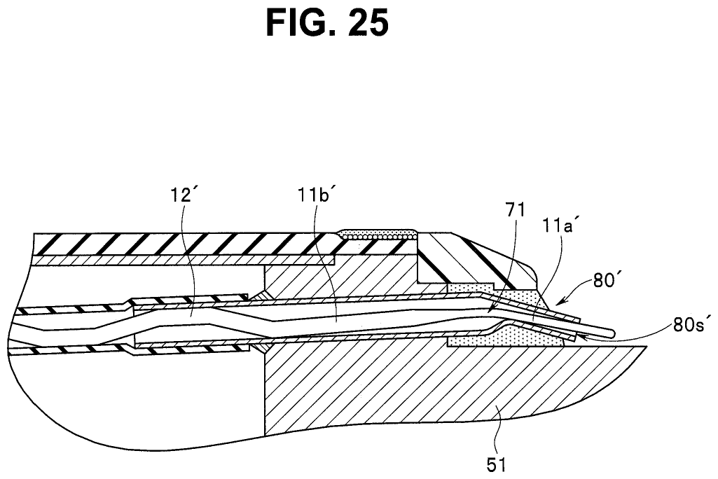

FIG. 24 is a partial cross-sectional view showing a state where the conduit cleaning tool in FIG. 21 is inserted through the gas feeding conduit or the liquid feeding conduit of the endoscope. FIG. 25 is an enlarged cross-sectional view of the part enclosed by the line IIXV in FIG. 24.

As shown in FIG. 24 and FIG. 25, in a structure in which a nozzle 80′ cannot be detached in the distal end opening 71 of the gas feeding conduit 62, unlike the above-described first to third embodiments, since an opening 80s′ of the nozzle 80′ is extremely small, there is a case where it is difficult to insert the shaft 10 into the gas feeding conduit 62 or the liquid feeding conduit 63 through the distal end opening 71.

In such a case, as shown in FIGS. 21 to 23, in the first part 11′ of the shaft 10′ of the conduit cleaning tool 1′ shown in the present embodiment, a distal end side part 11a′ in the longitudinal direction N may be formed to be thinner than a proximal end side part 11b′ in the bent direction Y (t<T).

If the conduit cleaning tool 1′ shown in FIGS. 21 to 23 is used to clean the inside of the gas feeding conduit 62 or the inside of the liquid feeding conduit 63 of the endoscope to which the nozzle 80′ is fixed as shown in FIG. 24 and FIG. 25, if an insertion assisting instrument 400 mounted in the fitting hole 66 is used to insert the conduit cleaning tool 1′ selectively into the gas feeding conduit 62 or the liquid feeding conduit 63 as shown in FIG. 24 and FIG. 25, the same effects as those in the above-described present embodiment can be obtained.

In addition, with regard to the gas feeding conduit 62, if the distal end side part 11a′ formed so as to have a small diameter is inserted until the distal end side part 11a′ protrudes from the distal end opening 80s′ of the nozzle 80′, even the inside of the nozzle 80′ can be surely scrubbed with the distal end side part 11a′, although it has been difficult to insert the first part 11′ of the present embodiment into the nozzle 80′, due to the diameter size of the first part 11′.

Note that other configurations and effects are the same as those in the above-described present embodiment.

Note that, in the above-described first to third embodiments, description has been made on the endoscope by taking the side-view endoscope as an example. However, it is needless to say that the present invention is not limited to the side-view endoscope but applicable also to a conduit of a front-view endoscope.

Furthermore, in the above-described first to third embodiments, description has been made on the conduit to be cleaned by using the conduit cleaning tool, by taking the gas feeding conduit 62 or the liquid feeding conduit 63 as an example. However, it is needless to say that the present invention is not limited to the same, but is applicable for cleaning another channel of the endoscope, such as the treatment instrument insertion channel 61 or the like.

Furthermore, the present invention is not limited to the above-described embodiments, but can be changed appropriately within a range not departing from the gist or concept of the invention that can be read from claims, throughout the specification, and the drawings.

What is claimed is:

1. An endoscope conduit cleaning tool comprising,
a long shaft having elasticity, the shaft being configured to be inserted into a conduit of an endoscope so as to be advanceable and retractable, and rotatable with respect to the conduit,
wherein the shaft comprises:
a first part arranged on a distal end side of the shaft in a longitudinal direction of the shaft, and formed in a linear shape so as to extend along the longitudinal direction; and
a second part arranged on a proximal end side of the shaft in the longitudinal direction with respect to the first part, and the second part including a plurality of bent portions bent to a corrugated shape in a predetermined direction with respect to the longitudinal direction, the plurality of bent portions being configured to rub an inner surface of the conduit.

2. The endoscope conduit cleaning tool according to claim 1, wherein the plurality of bent portions are shaped to displace the shaft in the predetermined direction so as to be corrugated with a predetermined depth in at least one plane.

3. The endoscope conduit cleaning tool according to claim 2, wherein a maximum value of the predetermined depth is larger than a maximum inner diameter in an entire length of the conduit.

4. The endoscope conduit cleaning tool according to claim 1, wherein a thickness of the second part of the shaft is set such that a thickness in another direction orthogonal to the predetermined direction and the longitudinal direction is larger than a thickness in the predetermined direction.

5. The endoscope conduit cleaning tool according to claim 1, wherein, in the first part of the shaft, a diameter of a distal end side in the longitudinal direction is formed to be smaller than a diameter of a proximal end side in the longitudinal direction.

6. The endoscope conduit cleaning tool according to claim 1, wherein the shaft is configured to be insertable into the conduit by using an insertion assisting instrument.

7. The endoscope conduit cleaning tool according to claim 1, wherein the shaft is insertable into and extractable from the conduit through a fluid discharge port of the conduit, the fluid discharge port opening at a distal end of an insertion portion of the endoscope.

8. The endoscope conduit cleaning tool according to claim 1, wherein the conduit is at least one of a gas feeding conduit and a liquid feeding conduit of the endoscope.

9. The endoscope conduit cleaning tool according to claim 1, wherein also the first part is further configured to rub the inner surface of the conduit.

10. An endoscope system comprising:
an endoscope conduit cleaning tool; and
an endoscope including a conduit into which the endoscope conduit cleaning tool is insertable,
the conduit cleaning tool comprising:
a long shaft having elasticity, the shaft being configured to be inserted into the conduit of the endoscope so as to be advanceable and retractable, and rotatable with respect to the conduit,
wherein the shaft comprises:
a first part arranged on a distal end side of the shaft in a longitudinal direction of the shaft, and formed in a linear shape so as to extend along the longitudinal direction; and
a second part arranged on a proximal end side of the shaft in the longitudinal direction with respect to the first part, and including a plurality of bent portions bent to a corrugated shape in a predetermined direction with respect to the longitudinal direction, the plurality of bent portions being configured to rub an inner surface of the conduit.

11. The endoscope system according to claim 10, further comprising
an insertion assisting instrument configured to guide the conduit cleaning tool into the conduit.
